(19) United States
(12) Patent Application Publication
(10) Pub. No.: US 2017/0116155 A1
(43) Pub. Date: Apr. 27, 2017

(54) SYSTEM AND METHOD OF CONJUGATE ADAPTIVE CONJUGATE MASKING EMPIRICAL MODE DECOMPOSITION

(71) Applicant: NATIONAL CENTRAL UNIVERSITY, TAOYUAN CITY (TW)

(72) Inventors: NORDEN E. HUANG, TAOYUAN CITY (TW); ZHAO-HUA WU, LAUREL, MD (US); JIA-RONG YEH, TAOYUAN CITY (TW)

(21) Appl. No.: 15/016,022

(22) Filed: Feb. 4, 2016

(30) Foreign Application Priority Data
Oct. 22, 2015 (CN) 201510688238.2

(51) Int. Cl. G06F 17/11; A61B 5/00; A61B 5/021

(52) U.S. Cl. CPC G06F 17/11; A61B 5/021; A61B 5/7271

(57) ABSTRACT

The invention discloses a method and a system for quickly and directly processing an original signal into a plurality of mode functions, the steps comprises: decomposing the original signal by Empirical Mode Decomposition (EMD) method to choose a first intrinsic mode functions (IMFs). Then, adding the plurality of level n conjugate masking functions, which are selected from a group of sinusoidal functions comprising the mean amplitude and the mean frequency of the first IMF, to the original signal individually to obtain level n mode functions, until the level n mode function is a monotonic function, wherein the plurality of mode functions are the IMFs between each frequency regions of the original signal. The invention not only includes the advantage of EMD analyzing, but also excludes the problem of mode mixing phenomenon which is caused by the intermittent disturbance.

### CROSS-REFERENCE TO RELATED APPLICATIONS

This Non-provisional application claims priority under 35 U.S.C. §119(a) on Patent Application No(s). [201510688238.2] filed in China [Oct. 22, 2015], the entire contents of which are hereby incorporated by reference.

### FIELD OF THE INVENTION

The invention relates to a method and system for low computation cost and no noise containment to decompose the nonlinear and non-stationary signal. More particularly, to a method and system of conjugate adaptive Conjugate Masking Empirical Mode Decomposition (CADM EMD) based on dyadic decomposition.

### BACKGROUND OF THE INVENTION

The Empirical Mode Decomposition (EMD) method based on an algorithm of Hilbert-Huang Transformation (HHT) is a versatile method for analyzing nonlinear and non-stationary data. The EMD method has been widely applied to many fields that including ocean waves, geophysics, climate changes, infrastructural health monitoring, machine vibrations and stability, biomedical sciences.

During EMD decomposing, the original signal based on high-frequency to low-frequency with corresponding functions in the form of the Intrinsic Mode Functions (IMFs). However, one of the main drawbacks of EMD is mode mixing. The mode mixing, which is the phenomenon of having oscillations of different scales residing in one IMF, thus it is impossible to assign clear physical meaning to that particular IMF component. The mode mixing phenomenon is the consequence of the intermittency in data resulting from the complex physical processes.

The Ensemble Empirical Mode Decomposition (EEMD) is presented (Wu and Huang, 2009). The key idea on the EEMD relies on averaging the modes obtained by EMD applied to several realizations of Gaussian white noise added to the original signal. The resulting solves the EMD mode mixing problem. However, the drawback of this statistically sound approach is mainly the computation cost. To get statistically significant mean without noticeable residual noise, the usual implement of the EEMD had to include more than 100 trials that means a hundred times slower than the EMD. Another drawback is the computation cost, which increase with the number of variables involved. Under this condition, the computation cost may be too high to be worthy of practical applications.

### SUMMARY OF THE INVENTION

The present invention provides to a method and a system of conjugate adaptive dyadic masking empirical mode decomposition (CADM EMD). CADM EMD not only includes the advantage of Empirical Mode Decomposition (EMD) analyzing, but also excludes the problem of mode mixing phenomenon which is caused by the intermittent disturbance.

In an embodiment of the invention, the present invention provides a method implemented in computer for processing an original signal into a plurality of mode functions, the method comprising: receiving the original signal; decomposing the original signal by EMD method to generate a plurality of intrinsic mode functions (IMFs); choosing a first IMF, then meaning each instant frequency and each instant amplitude of the first IMF to determine a mean frequency and a mean amplitude, wherein the first IMF is determined by a highest frequency region of the plurality of IMFs.

And adding a plurality of level one conjugate masking functions individually to the original signal to generate a plurality of level one modify signals, wherein the plurality of level one conjugate masking functions with same phase difference are selected from a group of sinusoidal functions comprising the mean amplitude and the mean frequency. The method further obtains a plurality of level one modify IMFs by EMD method to decompose the plurality of level one modify signals; summing the plurality of level one modify IMFs, and dividing by a number of level one conjugate masking function to obtain a level one mode function.

And adding a plurality of level two conjugate masking functions individually to the original signal to obtain a plurality of level two modify signals, wherein the plurality of level two conjugate masking functions with same phase difference are selected from a group of sinusoidal functions comprising the mean amplitude and the mean frequency; The method further obtains a plurality of level two modify IMFs by EMD method to decompose the plurality of level two modify signals: summing the plurality of level two modify IMFs, and dividing by a number of level two conjugate masking function to obtain a level two mode function.

Repeating steps above, adding a plurality of level n conjugate masking functions to the original signal individually to obtain a level n mode functions, until the level n mode function is a monotonic function, wherein the plurality of level n conjugate masking functions with same phase difference are selected from a group of sinusoidal functions comprising the mean amplitude and the mean frequency.

Finally, constructing the level one mode function to the level n mode function, wherein the mode functions are the IMFs between each frequency region of the original signal.

In another embodiment of the invention, the signal processing system comprises an input device, a computing device, and an output device.

The input device receives an original signal.

The computing device comprises a decomposing processor and an analyzing processor. The decomposing processor is connected to the input device for decomposing the original signal by EMD method to generate a plurality of IMFs, and choosing a first IMF, meaning each instant frequency and each instant amplitude of the first IMF to determine a mean frequency and a mean amplitude, wherein the first IMF is determined by a highest frequency region of the plurality of IMFs.

The analyzing processor is connected to the decomposing processor for adding a plurality of level one conjugate masking functions individually to the original signal to generate a plurality of level one modify signals, wherein the plurality of level one conjugate masking functions with same phase difference are selected from a group of sinusoidal functions comprising the mean amplitude and the mean frequency; then obtaining a plurality of level one IMFs by EMD method to decompose the plurality of level one modify signals; summing the plurality of level one modify IMFs, and dividing by a number of level one conjugate masking function to obtain a level one mode function.

Then adding a plurality of level two conjugate masking functions individually to the original signal to obtain a plurality of level two modify signals, wherein the plurality of level two conjugate masking functions with same phase difference are selected from a group of following sinusoidal functions comprising the mean amplitude and the mean frequency; then obtaining a plurality of level two modify IMFs by EMD method to decompose the plurality of level two modify signals: summing the plurality of level two modify IMFs, and dividing by a number of level two conjugate masking function to obtain a level two mode function.

Furthermore, repeating steps above, adding a plurality of level n conjugate masking functions to the original signal individually to obtain a level n mode function, until the level n mode function is a monotonic function, wherein the level n conjugate masking functions with same phase difference are selected from a group of sinusoidal functions comprising the mean amplitude and the mean frequency.

The output device is connected to the analyzing processor for constructing the level one mode function to the level n mode function, wherein the mode functions are the IMFs between each frequency region of the original signal.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views. The patent or application file contains at least one drawing executed in color. Copies of this patent or patent application publication with color drawing(s) will be provided by the Office upon request and payment of the necessary fee.

FIG. 1 is a block diagram of a system implemented in computer for processing an original signal into a plurality of mode functions.

FIGS. 2A and 2B illustrates the intrinsic mode functions (IMFs) decomposed by Empirical Mode Decomposition (EMD) and Ensemble Empirical Mode Decomposition (EEMD) in the prior art.

FIG. 3A and FIG. 3B illustrates the IMFs decomposed by Conjugate Adaptive Dyadic Masking EMD (CADM EMD) with phase difference π/4 (4 masking) and π/8 (8 masking) in the present disclosure.

FIG. 4A, illustrates the IMFs of a blood pressure signal decomposed by EEMD in the prior art.

FIG. 4B illustrates the intrinsic mode functions of a blood pressure signal decomposed by CADM EMD in the present disclosure.

FIG. 5 is a steps flowchart of a method implemented in computer for processing an original signal into a plurality of mode functions.

### DETAILED DESCRIPTION OF THE INVENTION

Having summarized various aspects of the present disclosure, reference will now be made in detail to the description of the disclosure as illustrated in the drawings. While the disclosure will be described in connection with these drawings, there is no intent to limit it to the embodiment or embodiments disclosed herein. On the contrary, the intent is to cover all alternatives, modifications and equivalents included within the spirit and scope of the disclosure as defined by the appended claims.

In an embodiment of the invention, the present invention discloses a method of conjugate adaptive conjugate masking empirical mode decomposition (CADM EMD) implemented in a signal analysis system during dyadic decomposition of nonlinear and non-stationary data in a low computation cost. It is understood that the method provides merely an example of the many different types of functional arraignments that may be employed to implement the operation of the various components of a system, for example, a computer system connected to a scanner, a multiprocessor computing device, and so forth. The execution steps of the present invention may include application specific software which may store in any portion or component of the memory including, for example, random access memory (RAM), read-only memory (ROM), hard drive, solid-state drive, magneto optical (MO), IC chip, USB flash drive, memory card, optical disc such as compact disc (CD) or digital versatile disc (DVD), or other memory components.

For some embodiments, the system comprises an input device, a computing device, and an output device. The input device used to receive data such as image, text or control signals and provide to a computing device such as a computer or other information appliance. In accordance with some embodiments, the computing device includes a storage medium and a plurality of processors. The storage medium is such as, by way of example and without limitation, a hard drive, an optical device or a remote database server coupled to a network, and stores software programs. The processors perform data calculations, data comparisons, and data copying. The output device that visually conveys text, graphics, and intrinsic mode function (IMF). Information shown on the output device is called soft copy because the information exists electronically and is displayed for a temporary period of time. The output device includes CRT monitors, LCD monitors and displays, gas plasma monitors, and televisions.

In accordance with such embodiments of present invention, the software programs are stored in the storage medium and executed by the processors when the computing device executes the method of CADM EMD. Finally, information provided by the computing device, and presented on the output device or stored in the storage medium.

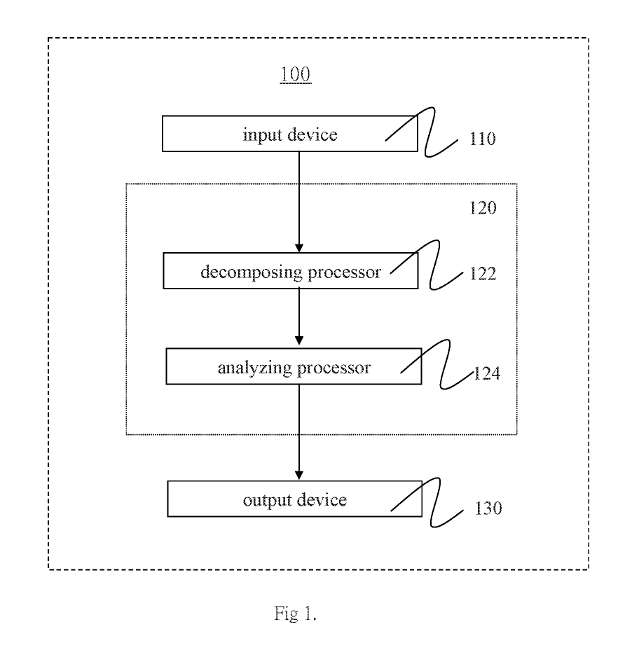

Please refer FIG. 1, it is understood that the method of conjugate adaptive dyadic masking EMD provides merely an example of the many different types of functional arrangements that may be employed to implement the operation of the various components of the signal processing system 100. The signal processing system 100 comprises an input device 110, a computing device 120 and an output device 130, wherein the computing device 120 comprises a decomposing processor 122, and an analyzing processor 124.

The input device 110 receives an input signal such as an original signal, wherein the original signal is a nonlinear and unstable signal (data). In another embodiment, the input device 110 may receive the original signal wirelessly.

The decomposing processor 122 is connected to the input device 110 decomposes the original signal by using an EMD method to generate a plurality of IMFs. The computing device 120 chooses a first IMF, meaning each instant frequency and each instant amplitude of the first IMF to determine a mean frequency (ωₒ) and a mean amplitude (aₒ), wherein the first IMF is determined by a highest frequency region of the plurality of IMFs.

The analyzing processor 124 is connected to the decomposing processor 122 for adding a plurality of level one conjugate masking functions individually to the original signal to generate a plurality of level one modify signals, wherein the plurality of level one conjugate masking functions with same phase difference are selected from a group of following sinusoidal functions comprising the mean amplitude and the mean frequency. And the analyzing processor 124 decomposes the level one modify signals by using the EMD method to obtain a plurality of level one modify IMFs.

In one embodiment, the level one conjugate masking functions are separated by phase difference of τ/2, wherein the level one conjugate masking function is computed based on the following formula:

+aₒ sin ωₒt, −aₒ sin ωₒt, +aₒ cos ωₒt and −aₒ cos ωₒt

In embodiment, the level one conjugate masking functions are separated by phase difference of π/4, wherein the level one conjugate masking function is computed based on the following formula:

+aₒ sin ωₒt, −aₒ sin ωₒt, +aₒ cos ωₒt, −aₒ cos ωₒt,

+aₒ sin(ωₒt+π/4), −aₒ sin(ωₒt+π/4), +aₒ cos(ωₒt+π/4) and −aₒ cos(ωₒt+π/4)

The level one conjugate masking function such as, by way of example and without limitation, π/8 or π/16 could be added as required by special situation, which will make the result even smoother, wherein the sinusoidal function further comprises (ωₒt+π/4) and (ωₒt+π/8). However, the computing time of the analyzing processor 124 is increasing when the phase difference is smaller. In an advantageous embodiment, the analyzing processor 124 performs a lower computation cost which has better performance than EMD and the EEMD when the phase difference is π/4. The level one modify IMFs of the invention are to make more accurate and guarantee conformation to the white noise characteristics.

The analyzing processor 124 further calculates the sum of the plurality of level one modify IMFs, and be divided by a number of level one conjugate masking function to obtain a level one mode function for elimination increasing progressively with level one modify IMFs, wherein the effects of the masking function will cancel out due to their paired conjugate properties of the masking functions, for example, sin and cos.

The analyzing processor 124 adds a plurality of level two conjugate masking functions individually to the original signal to obtain a plurality of level two modify signals, wherein the plurality of level two conjugate masking functions with same phase difference are selected from a group of sinusoidal functions comprising the mean amplitude (aₒ) and the mean frequency (ωₒ). And the analyzing processor 124 decomposes the plurality of level two modify signals by EMD method to obtain a plurality of level two modify IMFs.

In one embodiment, the level two conjugate masking functions are separated by phase difference of π/2, wherein the level two conjugate masking function is computed based on the following formula:

+(aₒ/2)sin(ωₒ/2)t,−(aₒ/2)sin(ωₒ/2)t,

+(aₒ/2)cos(ωₒ/2)t and −(aₒ/2)cos(ωₒ/2)t

In one embodiment, the level two conjugate masking functions are separated by phase difference of π/4, wherein the level two conjugate masking function is computed based on the following formula:

+(aₒ/2)sin(ωₒ/2)t, −(aₒ/2)sin(ωₒ/2)t,

+(aₒ/2)cos(ωₒ/2)t,−(aₒ/2)cos(ωₒ/2)t,

+(aₒ/2)sin(ωₒ/2+π/4)t, −(aₒ/2)sin(ωₒ/2+π/4)t,

+(aₒ/2)cos(ωₒ/2+π/4)t and −(aₒ/2)cos(ωₒ/2+π/4)t

The conjugate masking function such as, by way of example and without limitation, π/8 or π/16 could be added as required by special situation, which will make the result even smoother, wherein the sinusoidal function further comprises (ωₒt+π/4) and (ωₒt+π/8).

The analyzing processor 124 further calculates the sum of the plurality of level two IMFs, and be divided by a number of level two conjugate function to obtain a level two mode function. The level two conjugate masking functions with same phase difference are selected from a group of sinusoidal functions, for example, the mean amplitude (aₒ) and the mean frequency (ωₒ).

In one embodiment, the level two conjugate masking functions are separated by phase difference of π/2. The analyzing processor 124 performs steps above repeatedly for adding a plurality of level n conjugate masking functions to the original signal individually to obtain a level n mode function, until the level n mode function is a monotonic function from which no more mode function can be extracted. The plurality of level n conjugate masking functions with same phase difference are selected from a group of sinusoidal functions, for example, the mean amplitude (aₒ) and the mean frequency (ωₒ).

In one embodiment, the plurality of level n conjugate masking functions are separated by phase difference of π/2, wherein the level n conjugate masking function is computed based on the following formula:

+(aₒ/2ⁿ⁻¹)sin(ωₒ/2ⁿ⁻¹)t, −(aₒ/2ⁿ⁻¹)sin(ωₒ/2ⁿ⁻¹)t,

+(aₒ/2ⁿ⁻¹)cos(ωₒ/2ⁿ⁻¹)t and −(aₒ/2ⁿ⁻¹)cos(ωₒ/2ⁿ⁻¹)t

In one embodiment, the plurality of level n conjugate masking functions are separated by phase difference of π/4, wherein the level n conjugate masking function is computed based on the following formula:

+(aₒ/2ⁿ⁻¹)sin(ωₒ/2ⁿ⁻¹)t, −(aₒ/2ⁿ⁻¹)sin(ωₒ/2ⁿ⁻¹)t,

+(aₒ/2ⁿ⁻¹)cos(ωₒ/2ⁿ⁻¹)t,−(aₒ/2ⁿ⁻¹)cos(ωₒ/2ⁿ⁻¹)t,

+(aₒ/2ⁿ⁻¹)sin(ωₒ/2ⁿ⁻¹+π/4)t, −(aₒ/2ⁿ⁻¹)sin(ωₒ/2ⁿ⁻¹+π/4)t,

+(aₒ/2ⁿ⁻¹)cos(ωₒ/2ⁿ⁻¹+π/4)t and −(aₒ/2ⁿ⁻¹)cos(ωₒ/2ⁿ⁻¹+π/4)t,

The conjugate masking function such as, by way of example and without limitation, π/8 or π/16 could be added as required by special situation, which will make the result even smoother, wherein the sinusoidal function further comprises (ωₒ/2ⁿ⁻¹+π/4) and (ωₒ/2ⁿ⁻¹+π/8).

The output device 130 is connected to the analyzing processor 124 for constructing the level one mode function to level n mode function, wherein the mode functions are the IMFs between each frequency regions of the original signal.

The output device 130 is visually conveys text, graphics, and the signal. Information shown on the output device 130 is called soft copy because the information exists electronically and is displayed for a temporary period of time. The output device 130 is connected to the system 100 including a touch screen device, a printer device, CRT monitors, LCD monitors and displays, gas plasma monitors, and televisions. For example, the touch screen device receives the nonlinear and non-stationary signal as an input signal from the system 100, for example, a computer or a signal analysis device. The nonlinear and non-stationary signal is decomposed by the EMD method to generate the IMFs. The IMF is displayed on the touch screen such as cardiogram or an image for user analysis and observation.

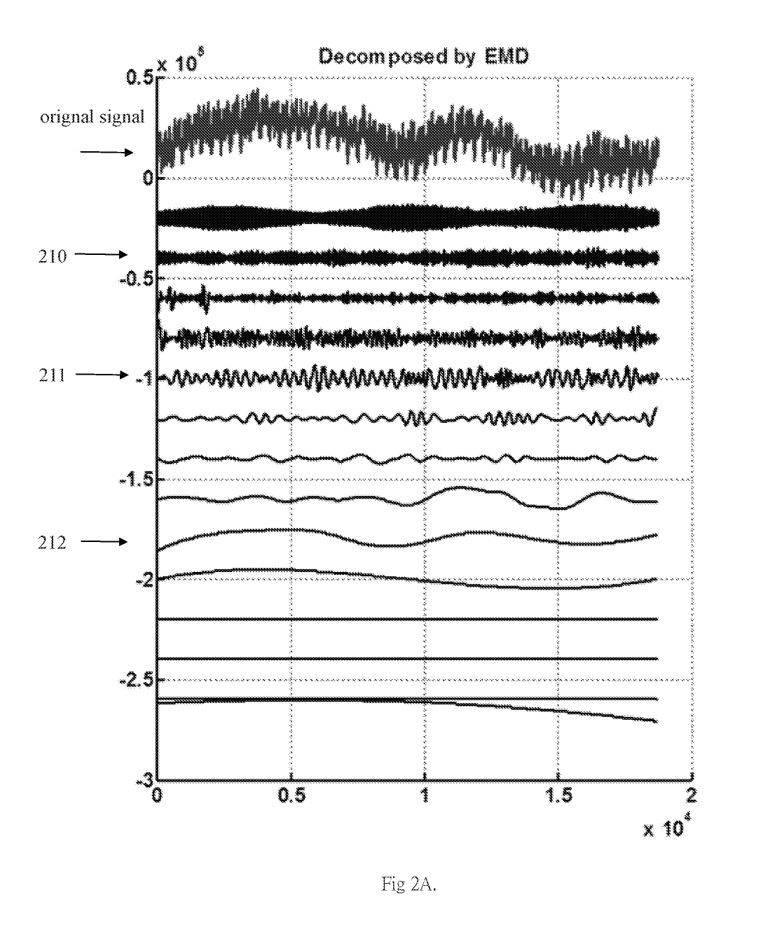

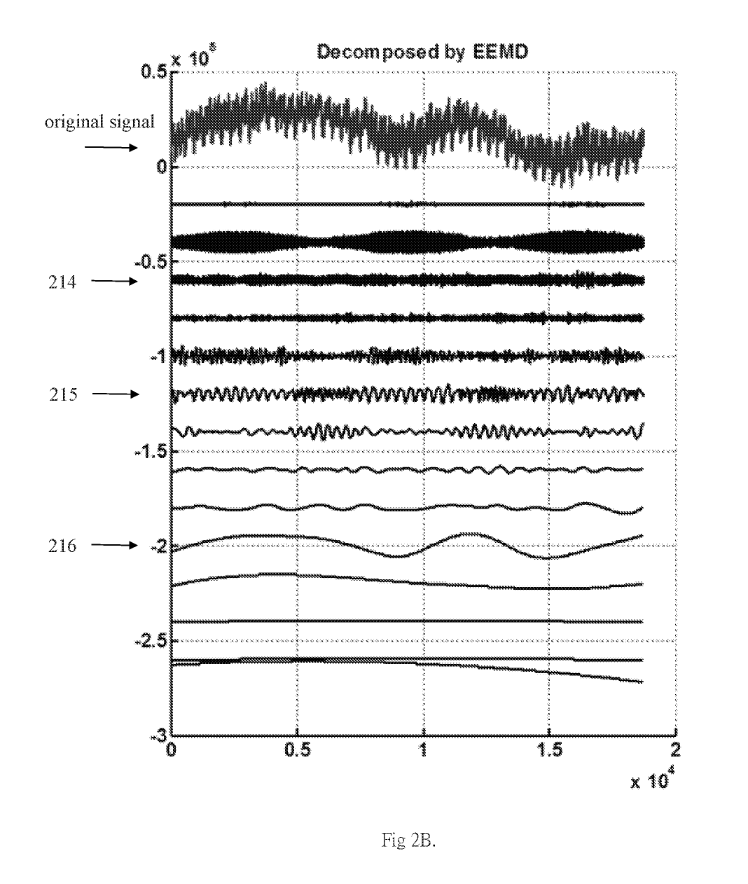

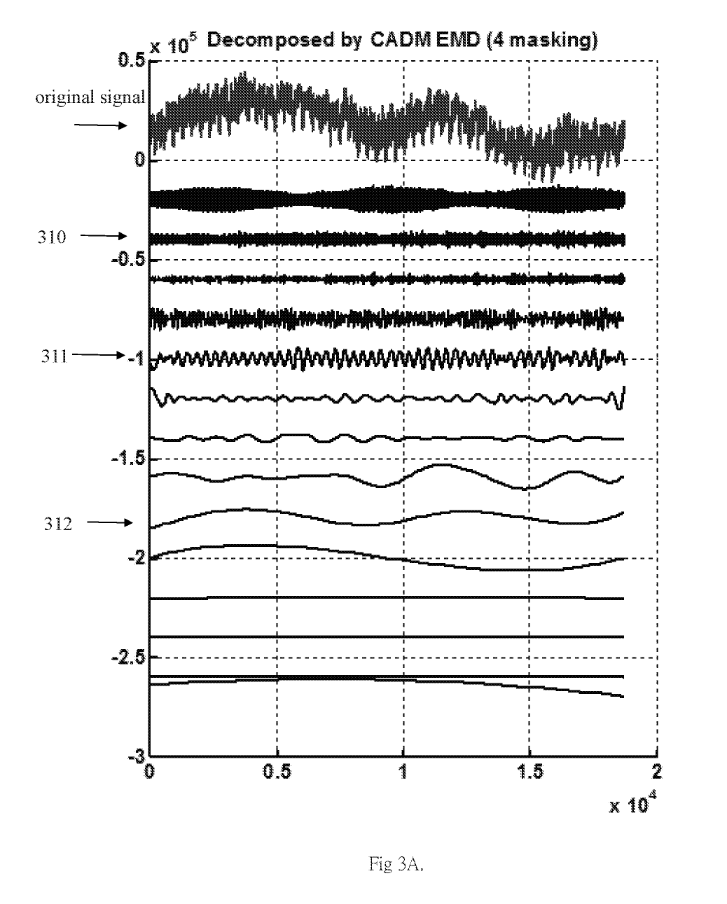

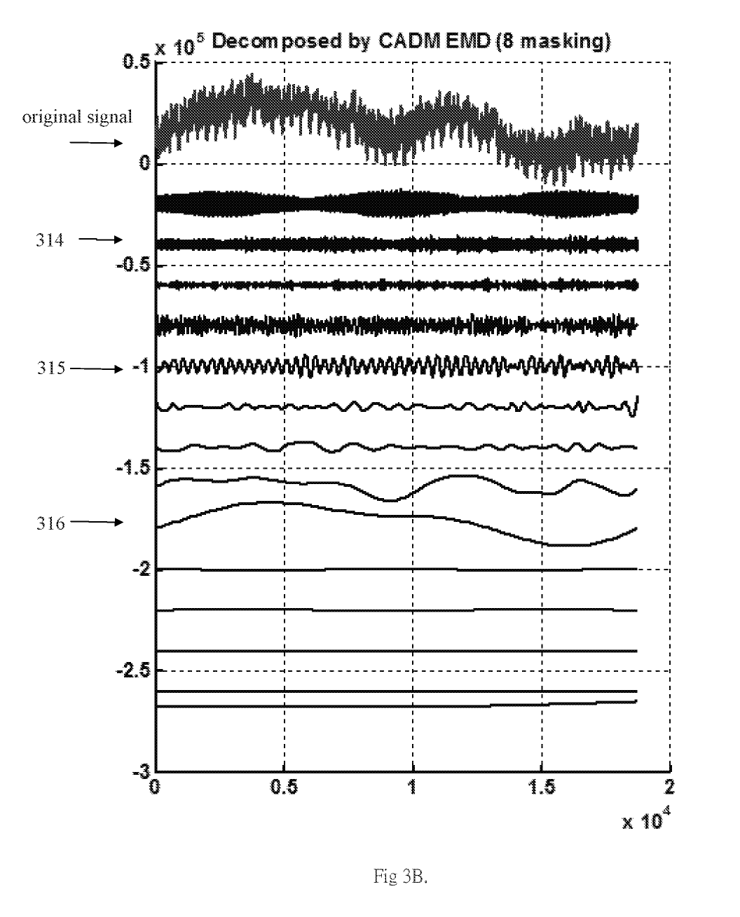

FIG. 2A and FIG. 2B illustrate the IMFs decomposed by EMD and EEMD in the prior art. FIG. 3A and FIG. 3B illustrate the IMFs decomposed by CADM EMD with phase difference π/4 (4 masking) and π/8 (8 masking) in the present disclosure. In FIG. 3A, the IMFs are generated based on the conjugate masking functions separated by the phase difference of π/2. In FIG. 3B, the IMFs are generated based on the conjugate masking functions separated by the phase difference of π/4. In FIGS. 2A, 2B, 3A, 3B, 4A and 4B illustrate the frequency regions 210, 211, 212, 214, 215, 216, 310, 311, 312, 214, 315, 316, 410, 412, 420, 422 are IMFs decomposed by EMD, EEMD or CADM EMD.

Reference is made to FIGS. 2A, 2B. 3A and 3B, the higher frequency regions 210, 214, 310, 314 are compared. As a result, the IMFs of the prior arts and the invention reflect uniformity characteristics with subtle difference frequencies and amplitudes. Further, the lower frequency regions 211, 215, 311, 315 are compared. As a result, the IMFs are generated by EMD. EEMD and CADM EMD which reflect different characteristics.

The invention eliminates inconsistent characteristic in the IMF to solve mode mixing problem. Furthermore, the lowest frequency regions 212, 216, 312, 316 are compared. As a result, the white noise is eliminated in the lowest IMFs based on CADM EMD method to solve mode mixing problem for influence frequencies and amplitudes. Therefore, the invention is adaptive, direct and noise free dyadic filter bank that produces clean IMFs for further analysis. The CADM EMD performs much better with no noticeable mode mixing in any IMF component. Furthermore, the scales in each IMF become more uniformly distributed. All the better results are obtained with much lower computational cost.

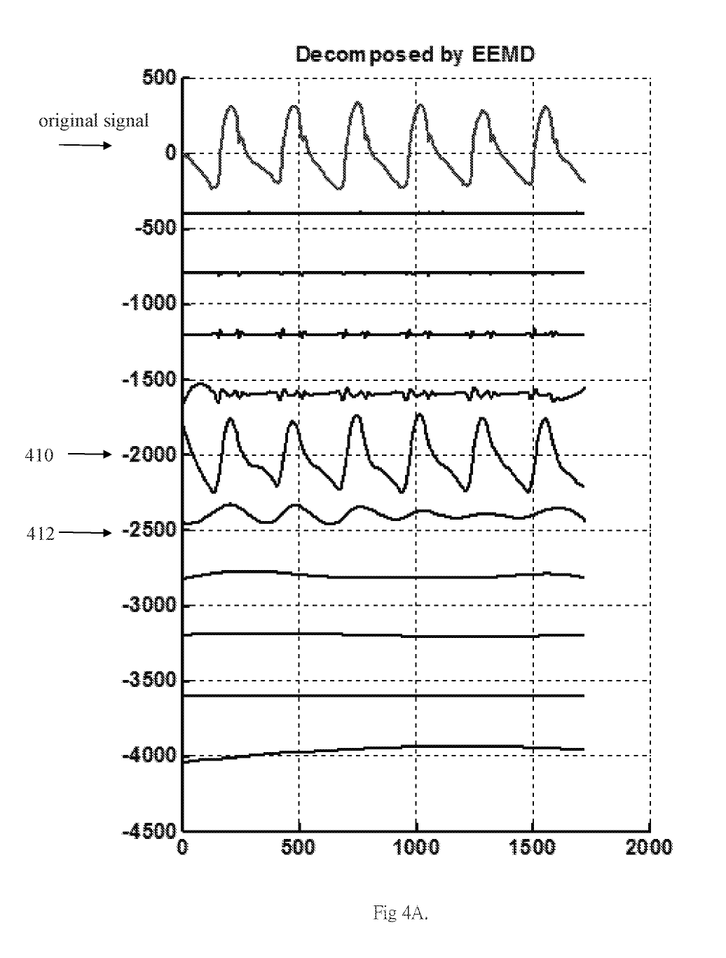

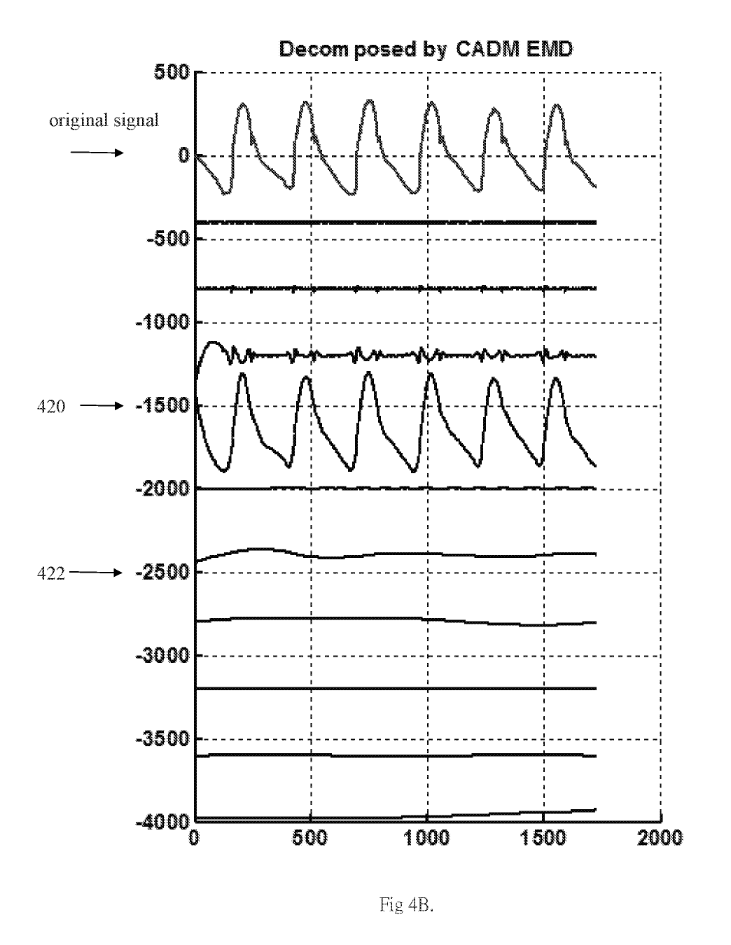

FIG. 4A, illustrates the IMFs of a blood pressure signal decomposed by EEMD in the prior art. FIG. 4B illustrates the IMFs of a blood pressure signal decomposed by CADM EMD in the present disclosure. Reference is made to FIG. 4A, the IMF 410 is decomposed by EEMD in the frequency region −1500 to −2000. The IMF 410 is mixed with notice signal, therefore a component of a similar scale residing in the IMF 410 and the IMF 420. The IMF 420 is decomposed by the CADM EMD which results showed regulation and complete of the IMF 420 without white notice influence.

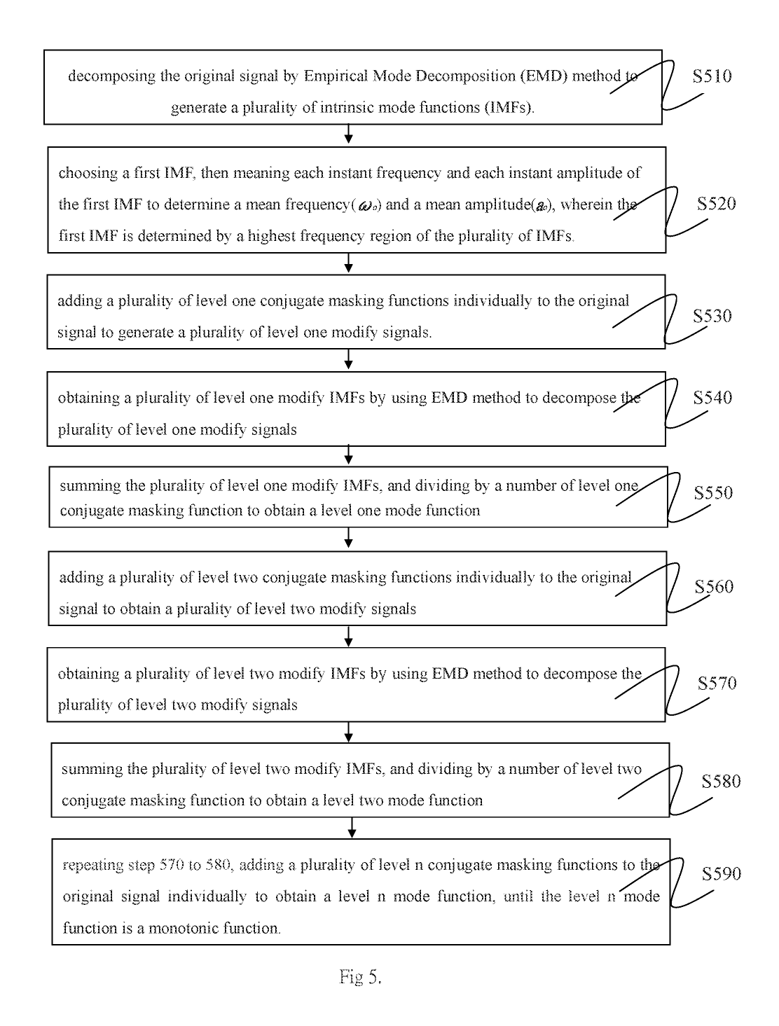

With further reference to FIG. 5, FIG. 5 is a flowchart of exemplary steps of a method of CADM EMD in accordance with various embodiments of the present disclosure. An original signal, for example, nonlinear and unstable signal (data) is obtained. Beginning with step 510, the original signal is decomposed by the EMD method to obtain the IMFs.

In step 520, the decomposing processor 122 chooses a first IMF, meaning each instant frequency and each instant amplitude of the first IMF to determine a mean frequency (ωₒ) and a mean amplitude (aₒ), wherein the first IMF is determined by a highest frequency region of the plurality of IMFs.

In step 530, the analyzing processor 124 adds the paired regular sine waves with mean amplitude (aₒ) and mean frequency (ωₒ) with same phase difference as conjugate masking functions to the original signal individual to generate a plurality of level one modify signals. The conjugate masking functions with finer phase distribution is implemented based on the following formula:

+aₒ sin ωₒt, −aₒ sin ωₒt, +ₐ Cos ωₒt and −aₒ cos ωₒt

In step 540, the analyzing processor 124 obtains a plurality of level one modify IMFs by using the EMD method to decompose the plurality of level one modify signals.

In step 550, the analyzing processor 124 sums the plurality of level one modify IMFs, and be divided by a number of level one conjugate masking function, to obtain a level one mode function.

In step 560, the analyzing processor 124 adds a plurality of level two conjugate masking functions individually to the original signal to obtain a plurality of level two modify signals. The level two conjugate masking functions with finer phase distribution is implemented based on the following formula:

+(ao/2)sin(ω/2)t, −(ao/2)sin(ωo/2)t,

+(ao/2)cos(ωo/2)t and −(ao/2)cos(ωo/2)t

In step 570, the analyzing processor 124 decomposes the plurality of level two modify signals by using the EMD method to obtain a plurality of level two IMFs.

In step 580, the analyzing processor 124 sums the plurality of level two IMFs, and be divided by a number of level two conjugate function to obtain a level two mode function.

In step 590, the analyzing processor 124 performs step 570 to 590 repeatedly. The analyzing processor 124 adds a plurality of level n conjugate masking functions individually to the original signal to obtain level n mode functions, until the level n mode function is a monotonic function from which no more mode function can be extracted. The level one conjugate masking function is computed based on the following formula:

+(aₒ2ⁿ⁻¹)sin(ωₒ/2ⁿ⁻¹)t, −(aₒ/2ⁿ⁻¹)sin(ωₒ/2ⁿ⁻¹)t,

+(aₒ/2ⁿ⁻¹)cos(ωₒ/2ⁿ⁻¹)t [symbol] −(aₒ/2ⁿ⁻¹)cos(ωₒ/2ⁿ⁻¹)t

Finally, the constructor unit 140 records the level one mode function to level n mode function, wherein the mode functions are the IMFs between each frequency regions of the original signal.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

What is claimed is:

1. A method implemented in computer for processing an original signal into a plurality of mode functions, the method comprising:
(A) receiving the original signal;
(B) decomposing the original signal by Empirical Mode Decomposition (EMD) method to generate a plurality of intrinsic mode functions (IMFs);
(C) choosing a first IMF, then meaning each instant frequency and each instant amplitude of the first IMF to determine a mean frequency (ωₒ) and a mean amplitude (aₒ), wherein the first IMF is determined by a highest frequency region of the plurality of IMFs;
(D) adding a plurality of level one conjugate masking functions individually to the original signal to generate a plurality of level one modify signals, wherein the plurality of level one conjugate masking functions with same phase difference are selected from a group of following sinusoidal functions comprising the mean amplitude (aₒ) and the mean frequency (ωₒ):
+aₒ sin ωₒt, −aₒ sin ωₒt, +aₒ cos ωₒt and −aₒ cos ωₒt
(E) obtaining a plurality of level one modify IMFs by using EMD method to decompose the plurality of level one modify signals;
(F) summing the plurality of level one modify IMFs, and dividing by a number of level one conjugate masking function to obtain a level one mode function;
(G) adding a plurality of level two conjugate masking functions individually to the original signal to obtain a plurality of level two modify signals, wherein the plurality of level two conjugate masking functions with same phase difference are selected from a group of following sinusoidal functions comprising the mean amplitude (aₒ) and the mean frequency (ωₒ);
+(aₒ/2)sin(ωₒ/2)t, −(aₒ/2)sin(ωₒ/2)t,
+(aₒ/2)cos(ωₒ/2)t and −(aₒ/2)cos(ωₒ/2)t
(H) obtaining a plurality of level two modify IMFs by using EMD method to decompose the plurality of level two modify signals;
(I) summing the plurality of level two modify IMFs, and dividing by a number of level two conjugate masking function to obtain a level two mode function;
(J) repeating step (H) to (I), adding a plurality of level n conjugate masking functions to the original signal individually to obtain a level n mode function, until the level n mode function is a monotonic function, wherein the plurality of level n conjugate masking functions with same phase difference are selected from a group of following sinusoidal functions comprising the mean amplitude (aₒ) and the mean frequency (ωₒ); and
+(aₒ/2ⁿ⁻¹)sin(ωₒ/2ⁿ⁻¹)t, −(aₒ/2ⁿ⁻¹)sin(ωₒ/2ⁿ⁻¹)t,
+(aₒ/2ⁿ⁻¹)cos(ωₒ/2ⁿ⁻¹)t and −(aₒ/2ⁿ⁻¹)cos(ωₒ/2ⁿ⁻¹)t
(K) constructing the level one mode function to the level n mode function, wherein the mode functions are the IMFs between each frequency region of the original signal.

2. The method according to claim 1, wherein the plurality of frequencies and the plurality of amplitudes are determined by a section which is at the highest frequency oscillations of the first IMF.

3. The method according to claim 1, wherein the conjugate masking functions is separated by the phase difference of π/2.

4. The method according to claim 1, wherein the conjugate masking functions is separated by the phase difference of π/4.

5. A system for processing an original signal into a plurality of mode functions, comprising:
an input device, receiving the original signal:
a computing device, comprising:
a decomposing processor connected to the input device for decomposing the original signal by Empirical Mode Decomposition (EMD) method to generate a plurality of intrinsic mode functions (IMFs), and choosing a first IMF, meaning each instant frequency and each instant amplitude of the first IMF to determine a mean frequency (ωₒ) and a mean amplitude (aₒ), wherein the first IMF is determined by a highest frequency region of the plurality of IMFs; and
an analyzing processor connected to the decomposing processor for adding a plurality of level one conjugate masking functions individually to the original signal to generate a plurality of level one modify signals, wherein the plurality of level one conjugate masking functions with same phase difference are selected from a group of following sinusoidal functions comprising the mean amplitude (aₒ) and the mean frequency (ωₒ),
+aₒ sin ωₒt, −aₒ sin ωₒt, +aₒ cos ωₒt and −aₒ cos ωₒt
then obtaining a plurality of level one modify IMFs by using EMD method to decompose the plurality of level one modify signals, summing the plurality of level one modify IMFs, and dividing by a number of level one conjugate masking function to obtain a level one mode function, then adding a plurality of level two conjugate masking functions individually to the original signal to obtain a plurality of level two modify signals, wherein the plurality of level two conjugate masking functions with same phase difference are selected from a group of following sinusoidal functions comprising the mean amplitude (aₒ) and the mean frequency (ωₒ),
+(aₒ/2)sin(ωₒ/2)t,−(aₒ/2)sin(ωₒ/2)t,
+(aₒ/2)cos(ωₒ/2)t and −(aₒ/2)cos(ωₒ/2)t
then obtaining a plurality of level two modify IMFs by using EMD method to decompose the plurality of level two modify signals, summing the plurality of level two IMFs, and dividing by a number of level two conjugate masking function to obtain a level two mode function, furthermore repeating steps above, adding a plurality of level n conjugate masking functions to the original signal individually to obtain a level n mode function, until the level n mode function is a monotonic function, wherein the level n conjugate masking functions with same phase difference are selected from a group of following sinusoidal functions comprising the mean amplitude (aₒ) and the mean frequency (ωₒ); and
+(aₒ/2ⁿ⁻¹)sin(ωₒ/2ⁿ⁻¹)t, −(aₒ/2ⁿ⁻¹)sin(ωₒ/2ⁿ⁻¹)t,
+(aₒ/2ⁿ⁻¹)cos(ωₒ/2ⁿ⁻¹)t and −(aₒ/2ⁿ⁻¹)cos(ωₒ/2ⁿ⁻¹)t
an output device connected to the analyzing processor for constructing the level one mode function to the level n mode function, wherein the mode functions are the IMFs between each frequency region of the original signal.

6. The system of claim 5, wherein the processing processor is further to determine the plurality of frequencies and the plurality of amplitudes according to a section which is at the highest frequency oscillations of the first IMF.

7. The system of claim 5, wherein the conjugate masking functions is separated by the phase difference of π/2.

8. The system of claim 5, wherein the conjugate masking functions is separated by the phase difference of π/4.
